(19) United States
(12) Patent Application Publication
(10) Pub. No.: US 2026/0294973 A1
(43) Pub. Date: Oct. 1, 2026

(54) COMPOSITIONS AND METHODS FOR THE TREATMENT OF INTESTINAL INFLAMMATION

(71) Applicants: Northwestern University, Evanston, IL (US); Ann and Robert H. Lurie Children's Hospital of Chicago, Chicago, IL (US)

(72) Inventors: Arun Sharma, Evanston, IL (US); Matthew Bury, Chicago, IL (US); Tiffany Sharma, Chicago, IL (US); Muthukumar Gunasekaran, Evanston, IL (US); Sunjay Kaushal, Evanston, IL (US)

(21) Appl. No.: 18/880,350

(22) PCT Filed: Jul. 3, 2023
(86) PCT No.: PCT/US2023/069592

§ 371 Date: Dec. 31, 2024

Related U.S. Application Data
(60) Provisional application No. 63/367,557, filed on Jul. 1, 2022.

(51) Int. Cl. A61K 35/28; A61K 35/12; A61P 1/00; A61P 29/00; C12N 5/0775

(52) U.S. Cl. CPC A61K 35/28; A61P 1/00; A61P 29/00; C12N 5/0668; A61K 2035/124

(57) ABSTRACT

Disclosed herein are composition and methods for treating a subject having intestinal inflammation. The methods comprise administering an effective amount of c-Kit+CD45− mesenchymal stem cells to a subject in need of a treatment for intestinal inflammation.

### CROSS-REFERENCE TO RELATED APPLICATIONS

This application claims priority to U.S. Provisional Application No. 63/367,557 filed on Jul. 1, 2022, the contents of which are incorporated by reference in their entireties.

### STATEMENT REGARDING FEDERALLY SPONSORED RESEARCH

N/A

### SEQUENCE LISTING

N/A

### FIELD

The field of the disclosure relates to methods and compositions for treating intestinal inflammation.

### BACKGROUND

Inflammatory bowel disease (IBD) is a group of disorders, including Crohn's disease and ulcerative colitis, that cause chronic inflammation in the gastrointestinal tract. IBD is a chronic, progressive, relapsing and remitting disorder that leads to complication including bowel damage, hospitalization, surgery and decreased quality of life. Crohn's disease (CD) is in-part due to a highly pro-inflammatory lesion microenvironment whose progression is accompanied by numerous clinical complications.

Since Crohn's disease is a chronic and progressive disease, optimal treatment early in the disease course to prevent complications is imperative. Current treatment options for CD patients include biological/pharmacological anti-inflammatory therapies. Despite the availability of current medications, the rates of primary and secondary treatment failure remain high. For unknown reasons there are appreciable rates of primary non-response, loss of response, or adverse reactions to the therapy, thereby necessitating additional treatment options. Patients who are refractory to therapeutic treatment typically require surgical intervention. Approximately 70% of patients with CD will require surgery during their lifetime. Unfortunately, surgery does not cure CD and most patients will continue to need supportive therapy post-surgery. The lack of long-longstanding efficacious treatment modalities illustrates the need for novel therapeutic approaches to modulate intestinal inflammation and promote wound healing.

### SUMMARY

The present invention provides a composition for treating intestinal inflammation. The present invention further provides a method of treating intestinal inflammation with neonatal mesenchymal stem cells.

The present disclosure provides a method for decreasing gastrointestinal inflammation, the method comprising administering a therapeutically effective amount of c-Kit+CD45− mesenchymal stem cells to a subject to decrease intestinal inflammation. Suitably, the c-Kit+CD45− mesenchymal stem cells may be neonatal or adult in origin and the gastrointestinal inflammation may be due to inflammatory bowel disease, such as Crohn's disease.

In some embodiments, the method described herein may decrease TNFα and CD68 and/or increase IL-10 and CD206. In some embodiments, the method increases gastrointestinal transit time.

In some embodiment the present disclosure provides a method for treating inflammatory bowel disease, the method comprising administering to a subject a therapeutically effective amount of c-Kit+CD45− mesenchymal stem cells to treat inflammatory bowel disease.

In some embodiment, the method for treating gastrointestinal inflammation comprises the steps of (a) isolating mesenchymal stem cells from a subject, (b) selecting c-Kit+CD45− mesenchymal stem cells from the isolated cells, (c) expanding the mesenchymal stem cells in culture and (d) administering the expanded c-Kit+CD45− mesenchymal stem cells to the subject in an amount effective to treat gastrointestinal inflammation. Suitably, the stem cells may be frozen after step (b) or (c), or the administering step of (d) may be injecting the expanded c-Kit+CD45− mesenchymal stem cells into the inflammatory lesion of the subject.

One aspect of the present disclosure provides a composition comprising in vitro culture expanded c-Kit+CD45− mesenchymal stem cells capable of decreasing intestinal inflammation. Suitably, the composition may comprise at least 90% c-Kit+CD45− cells and/or a pharmaceutically acceptable carrier.

Another aspect of the present disclosure provides a method of reducing inflammatory cytokines and increase in anti-inflammatory IL-10 and CD206 in inflammatory lesions in a subject, the method comprising: administering an effective amount of c-Kit+CD45− mesenchymal stem cells to the subject to reduce inflammatory cytokines and increase IL-10. The method may comprise isolating and expanding c-Kit+CD45− MSC from the subject prior to administration. In particular embodiments, the inflammatory cytokines include TNF-α, INFγ, CD68, or combinations thereof, the lesions are gastrointestinal lesions, and the administration is a local injection of the cells into the lesions of the gastrointestinal tract.

### BRIEF DESCRIPTION OF THE DRAWINGS

FIG. 1. Phenotypic characterization of nMSCs. Flow cytometry analysis of nMSCs and aMSCs for stem cell specific surface markers (SSEA3, SSEA3, CD105, CD90, CD34, CD45), cardiac lineage markers (MHC, GATA4, NKX2.5, ISL1), and mast cell marker (tryptase) (n=4).

FIG. 2. Ileitis lesions injected with nMSCs. Lesions (5 lesions/animal) were independently injected once (1e5 nMSCs/lesion) with either nMSCS (DI nMSCs) or control media (DI Control). Lesion size pre/post-injections were evaluated.

FIG. 3. Cytokine expression from Ileitis lesions injected with nMSCs. Ileum sections were single stained with immunofluorescent antibodies for inflammatory cells CD68 (macrophages), and cytokines TNFa, IFNy, and IL-10. Quantitative morphometric analyses were performed on stained tissue sections using ImageJ (n=3 images/lesion).

FIG. 4. Effectiveness of CPCs to reduce intestinal inflammation in SAMP mice. (a). (i, ii) Tail vein (TV) injected CPCs (20×106 or 10×106 CPCs/kg, respectively demonstrated reduced number and size of skip lesions (green arrows). (iii) A TV dose of 1×106 CPCs/kg was not as effective at reducing skip lesions in both size and number at the gross level in which animals also displayed strictures (yellow arrows) that followed skip lesions. (iv) Media-injected SAMP mice displayed numerous skip lesions followed by strictures. (b). Physical measurements of skip lesion demonstrated a significant reduction in lesion size (~47%) with the 10×106 CPC/kg dose compared to untreated SAMP mice. n=4 animals/condition for i-iii and n=3 animals/condition for iv. n=6 lesions analyzed/animal for every group 5 weeks post-injection. p<0.0001.

FIG. 5. Decrease in pro-inflammatory ileum environment. A) IF staining of ileum objectively demonstrating a decrease in inflammatory cytokines and cells but with an upregulation of anti-inflammatory IL-10. B) and C) Quantitative evaluation of the aforementioned conditions corroborate IF staining data. For cell markers, ***p<0.001 and **p<0.01 for cytokines. n=4 animals for CPC treatment and n=3 animals for control. n=6 lesions/animal. Analyzed/animal for every group 5 weeks post-injection.

FIG. 6. Intestinal transit. Data demonstrate that CPC treated animals (solid lines) exhibited increased FITC-dextran transit compared to untreated animals (dashed line).

FIG. 7. Ileitis status following direct injection of nMSCs into SAMP mice: Representative images of intestinal tissue lesion at 5 weeks following a single injection of nMSCs in SAMP mice. Ileitis lesion wound healing was analyzed by histology following nMSCs administration and compared with non-injections and placebo controls (a, b). Data are represented as mean±SEM (n=3-4). Representative H&E staining images revealed reduced infiltration of inflammatory cells and cryptic damage within the submucosa and lamina propria in nMSC administered mice compared to no-injection and placebo controls (c). White arrows depict areas of inflammatory cell infiltrate. nMSC treated animals have a subjectively lower level of immune cell presence in lesion tissue. Immunofluorescence staining of nMSCs with anti-α4β7 and L-selectin (CD62L) antibodies demonstrating the expression of these pivotal proteins in gut-immune cell trafficking (d). Scale bars-10×-200 μm, 40×-50 μm. Statistical significance was analyzed by the Mann-Whitney U test and p<0.05 were considered as significant.

FIG. 8. The percentage of macrophage phenotypes following nMSCs administration: Representative images show the percentage of CD68, CD86 and CD206 cells measured by immune histochemistry using intestinal lesions following nMSCs treatment in SAMP mice (a, b). Magnification 40×, Scale bar 50 μm. The tissue lesion samples collected from the SAMP mice following direct injections of nMSCs were utilized to evaluate the nMSCs retention by targeting human leukocyte antigen by immune-histology (HLA-A; c) Magnification 20×, Scale bar 100 μm. The frequency of nMSC retention was expressed as a percentage of nMSCs in the lesion area compared to placebo control (d). 3-4 mice were studied in each group. Data are represented as mean and standard error of the mean of the percentage of CD68 and CD206 cells. Statistical significance was analyzed by the Mann-Whitney U test and p<0.05 were considered as significant.

FIG. 9. The level of pro-and anti-inflammatory cytokines in the lesions: Histological analysis of lesions tissues shows significant reduction in pro-inflammatory cytokines (IFN-y, TNF-α) and elevated anti-inflammatory cytokine IL-10 following nMSC injection compared to no-injection and placebo controls (a, b). Magnification 40×, scale bar 50 μm. 3-4 mice were studied in each group. Statistical significance was analyzed by the Mann-Whitney U test and p<0.05 were considered as significant.

FIG. 10. Therapeutic efficacy of nMSCs on GI transit delay and segment gross pathology scores in SAMP mice: GI transit was assessed at five weeks post-injection (15 W of age) using FITC-dextran distribution across the GI tract which revealed the significant GI transit improvement in small bowel (SB) region in mice injected with nMSCs compared to no-injection and placebo controls. The total of 15 total segments including stomach (11 segments) to colonic regions (4 segments) were measured and each segment was represented as percent of total fluorescence and the distribution weighted toward more proximal segments considered indicative of slowing of transit. The mean fluorescence intensity of SB was peaked at segments SB5-SB7 in no injection and placebo controls compared to nMSCs treated mice (FIG. 10). Conversely, the fluorescence intensity of nMSCs peaked at colonic region (segment SB8 to Col1). The cumulative fluorescence percentage of the SB3-SB8 region was comparatively lower in the nMSCs treated group compared to no injection and placebo. P values were calculated using one-way ANOVA with Dunnett's post-hoc test. Values are presented as means±SEM (n=3-4 animals per group). p<0.05 were considered as statistically significant.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION

The present disclosure describes a composition for treating intestinal inflammation as well as methods for making and using the same.

Crohn's disease (CD) is characterized by chronic inflammation with severe devolving intestinal physiology resulting in increased morbidity and mortality.¹ CD pathogenesis encompasses overactive innate and adaptive immune cell behavior, environmental and socio-economic factors, and an assortment of genetic mutations.^{2,3} Adaptive and innate immune cells release pro-inflammatory cytokines that exacerbate skip lesion growth typically within the small intestine but can affect all aspects of the gastrointestinal tract (GI).^{4,6} An armamentarium of medical treatments which include corticosteroids, aminosalicylates, anti-microbial agents, immune modulators, immune suppressive regimens, and antibody therapies have been deployed to treat patients with CD at various stages of disease progression.⁷⁻⁹ However, the development of treatment insensitivity along with multiple severe side effects due to combinatorial treatments decrease health related quality of life metrics while increasing patient morbidity including the increased risk of developing GI cancers.^{8,10,11} The lack of long-longstanding efficacious treatment modalities illustrates the need for novel cell therapeutic approaches to significantly modulate intestinal inflammation and promote wound healing.

As described herein, the inventors have demonstrated that direct injection of human cardiac derived nMSCs could alleviate systemic inflammation and recover intestinal physiology in a murine model of CD-like ileitis.

Neonatal mesenchymal stem cells (nMSCs) are a self-renewing population of adherent multipotent progenitor cells with the capacity to differentiate into several mesenchymal cell lineages including bone, cartilage and adipose tissue. MSC typically express CD105, CD73 and/or CD90 and do not typically express CD45, CD34, CD14, CD11b, CD79, CD19 or HLA-DR. MSC can also be characterized based on their origin such as bone marrow, umbilical cord, adipose, lung or cardiac mesenchymal stem cells. In the present disclosure MSC can also be called CPC (cardiac progenitor cell) and are characterized as c-Kit+CD45−. MSC possess anti-inflammatory and pro-regenerative characteristics. nMSCs can be harvested from patients then expanded and further banked for future use. nMSCs are also immune privileged and can bypass host immune reactions following delivery. The present disclosure uses these cells as a cellular therapeutic platform to positively modulate the localized inflammatory milieu, for example, as demonstrated in the examples for a mouse model of Crohn's disease-like ileitis. The nMSCs can be delivered either systemically or via direct lesion injection to combat inflammation with no known side effects as compared to some current therapies. Further, the nMSCs can be used clinically with great efficacy. In one embodiment, the disclosure provides a method of treating intestinal or gastrointestinal inflammation in a subject. The method comprises administering a therapeutically effective amount of c-Kit+CD45− mesenchymal stem cells to the subject to decrease intestinal inflammation.

Gastrointestinal and/or intestinal inflammation may include inflammation in any part of the gastrointestinal tract including mouth, pharynx, esophagus, stomach, small intestine, large intestine, rectum, and anus. Gastrointestinal inflammation may also include Inflammatory Bowel Disease (IBD). IBD is a group of inflammatory conditions of the colon and small intestine, Crohn's disease and ulcerative colitis being the principal types.

Crohn's disease (CD) is a type of IBD that may affect any segment of the gastrointestinal tract. Symptoms often include abdominal pain, diarrhea (which may be bloody if inflammation is severe), fever, abdominal distension, and weight loss. CD is in-part due to a highly pro-inflammatory lesion microenvironment whose progression is accompanied by numerous clinical complications. CD may also be called ileitis.

Gastrointestinal inflammation, IBD and CD are characterized by elevated inflammatory proteins or markers including but not limited to, C-reactive protein, IL-1, IL-6, IL-8, IL-11, IL-12, IL-18, TNFα, TNF-β, IL-17, IFNγ, IFN-α, IFN-β, IL-4, IL-13, IL-9, IL-21, IL-22, G-CSF, macrophages, monocytes, neutrophils, natural killer cells, activated T cells, CD68, including CD68+macrophages, IgG and fecal calprotectin. Intestinal inflammation may be in any region of the gastrointestinal track. A decrease in gastrointestinal inflammation or intestinal inflammation may include a decrease in any of the inflammatory markers included herein. A decrease in gastrointestinal or intestinal inflammation may also include an increase in anti-inflammatory proteins. Ant-inflammatory proteins generally work to control anti-inflammatory cytokines and promote healing. Anti-inflammatory proteins may include, but are not limited to IL-1RA, IL-4, IL-5, IL-10, IL-11, IL-12, IL-13 IL-22, IL-27, IL-35, IL-37, IL-38, TGFB, FoxP3, Treg cells, iTreg cells, Th17 cells and stem cells. In some instances, a single protein, cytokine or cell may be anti-inflammatory or pro-inflammatory depending on the environment, including location or other proteins present at the same time, for example a T cell can be pro or anti-inflammatory.

An inflammatory lesion is an area of increased inflammation or any abnormal tissue or change in any area of the gastrointestinal tract.

This disclosure provides methods for decreasing gastrointestinal or intestinal inflammation. In some embodiments, these methods include administering mesenchymal stem cells to decrease intestinal inflammation. The term “decrease” or the related terms “decreased,” “reduce” or “reduced” refers to a statistically significant decrease. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero). As used herein, “decreased” means an amount below the mean of the particular cytokine or group of cytokines or substance found in a subject prior to treatment. In some embodiments TNFα, IFNγ and CD68 will be decreased by treatment. In some embodiments the treatment will elevate anti-inflammatory cytokines. In some examples, IL-10 and/or CD206, including CD206+ macrophages, will be increased. Increased anti-inflammatory cytokines means an amount above the amount that was present prior to treatment.

In some embodiment, the disclosure provides methods and compositions to treat intestinal inflammation with mesenchymal stem cells (MSC) including c-Kit+CD45− MSC. MSC are also known as mesenchymal stromal cells or medicinal signaling cells are multipotent stromal cells that can differentiate into a variety of cell types, including osteoblasts (bone cells), chondrocytes (cartilage cells), myocytes (muscle cells) and adipocytes. MSC can be derived from adult or neonatal tissue. Neonatal tissue may include bone marrow, amniotic cells, placental tissues, cord blood or umbilical cord. Neonatal tissue may be one month of age or less. Adult tissue may include bone marrow, adipose tissue or peripheral blood. MSC generally express c-Kit CD105, CD90, Nkx2.5 on their cell surface, and do not expression SSEA3, SSEA4, GATA4, ISL1, MCH, CD34, CD45, tryptase or CD31 on their cell surface. Other surface markers on MSC may include, but are not limited to CD73, CD44 CD166, CD13, STRO-1, CD9, CD29, CD54, CD73, CD106, CD146 and HLA1.MSC are typically negative for CD14, CD31. CD34, CD11b, CD19, CD31, CD79, CD133 and CD144. The MSC used in the present disclosure are derived from cardiac tissue and are c-Kit⁺CD45⁻.

In some embodiments, the disclosure provides methods and composition to increase intestinal transit time. Transit time may be increased by preventing or limiting the formation of adhesions or pathofibrosis. Decreased intestinal transit as a result of tissue fibrosis exhibited in ileitis can lead to weight loss, nutrient malabsorption, prolonged intestinal transit times and growth perturbations.

MSC can be isolated from bone marrow, adipose tissue, umbilical cord tissue (Wharton's Jelly), amniotic fluid, placenta or fetal tissue. In some embodiments, MSC can be isolated from atrial appendages. Neonatal MSC (nMSC) are immune privileged and can bypass host immune reactions.

In some embodiments, c-Kit+CD45− MSC may be isolated from a subject, then expanded and banked. To expand, MSC may be cultured in growth media, recombinant human FGF-basic, L-glutathione and human erythropoietin for a sufficient time to expand the culture.

MSC used in the disclosed methods can be selected and expanded from isolated cells from a subject. c-Kit+CD45− markers can be used to select MSC by means known in the art. Among others, cells can be selected with immunomagnetic techniques, fluorescence-activated cell sorting techniques, density gradient centrifugation, immunodensity cell separation, sedimentation, adhesion, microfluidic cell separation, aptamer technology, buoyancy-activated cell sorting, complement depletion, laser capture microdissection, immunoguided laser capture microdissection, limiting dilution, or micromanipulation. The cells may be autogeneic, or allogeneic.

The method described herein comprises administering c-Kit+CD45− MSC to a subject in need thereof to reduce intestinal inflammation. The methods described herein can be administered via systemic delivery (e.g., IV, etc.) or by local administration to the inflammatory lesions or affected areas.

Suitable dosages of cells may be determined by one skilled in the art. The cells may be administered one or more times at a concentration in the range of about 1×10⁵ to about 1×10¹⁵ cells/kg body weight, for example, about 50 million, 40 million, 30 million, 20 million, 10 million, 5 million or any concentration in-between cells/kg. Disease severity and subject morbidity may be considered when determining dosage. MSC may be administered by injection. Additionally, cells may be injected by a pump, or seeded onto a carrier for sustained or extended release. This can be given at the same time as intestinal flare ups experienced by the subject. Cells may also be given as a preventative, pre-ileitis, at the onset of ileitis and/or while ileitis lesions are inflamed. In some embodiments, the intestinal inflammation is due to inflammatory bowel disease (IBD), or Crohn's disease and the subject is a subject having IBD or Crohn's disease.

As used herein, a “subject” may be interchangeable with “patient” or “individual” and means an animal, which may be a human or non-human animal, in need of treatment. In particular embodiments, the subject is a human subject. A “subject in need of treatment” may include a subject having a disease, disorder, or condition that may be characterized as gastrointestinal inflammation. For example, the subject may have IBD, or Crohn's disease.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass therapeutic administration. In some embodiments, the subject is responsive to therapy with one or more of the compounds disclosed herein in combination with one or more additional therapeutic agents. The term “treating” comprises reducing one or more symptom or characteristic associated with intestinal inflammation. For example, the one or more symptoms may include, but are not limited to, for example, abdominal pain, diarrhea, bloody stool, mucus in stool, gas and bloating, upset stomach, fever, abdominal distension, change in appetite, rectal pain, fatigue, nausea or vomiting, and weight loss. Treating may also include, but is not limited to, reducing size or severity of inflammatory lesions, decrease in the number of inflammatory lesions, reducing the amount or number of pro-inflammatory markers expressed in the lesions or subject, increase in the anti-inflammatory markers in the lesions or subject, improvement in IBD or Crohn's disease activity index score, and reduction in other characteristics associated with inflammatory lesions.

As used herein the term “effective amount” refers to the amount or dose of the compound that provides the desired effect. In some embodiments, the effective amount is the amount or dose of the composition or cells, upon single or multiple dose administration to the subject, which provides the desired effect in the subject under diagnosis or treatment. Suitably the desired effect may be reducing the amount of gastrointestinal inflammation.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances. The present disclosure further provides method of decreasing inflammatory cytokines within an inflammatory lesion, the method comprising administering an effective amount of the c-Kit+CD45− MCSs to decrease inflammatory cytokines within the lesion. The inflammatory cytokines that are decreased within a lesion can include, for example, but not limited to, a decrease in TNFα, IFNγ and/or CD68. The method may also increase expression of one or more anti-inflammatory cytokines, for example, increase IL-10 expression in the inflammatory lesions or CD206+ macrophages.

The present disclosure further provides a method for treating inflammatory bowel disease, the method comprising administering to the subject a therapeutically effective amount of c-Kit+CD45− mesenchymal stem cells to treat the inflammatory bowel disease.

The present disclosure also provides a method of treating gastrointestinal inflammation, comprising the steps of: (a) isolating mesenchymal stem cells from a subject; (b) selecting c-Kit+CD45− mesencymal stem cells from the isolated cells; (c) expanding the mesenchymal stem cells in culture; and (d) administering the expanded c-Kit+CD45− mesenchymal stem cells to the subject in an amount effective to treat the gastrointestinal inflammation.

Isolating MSC can be done by any means known in the art. By way of example and not limitation, isolating MSC may be performed by gradient centrifugation, immunomagnetic cell separation, fluorescence-activated cell sorting, immunodensity cell isolation, microfluidic cell sorting or by adherence to plastic. Isolated MSC may be expanded in culture. Expansion may increase the number of MSC.

In some embodiments, a portion of the mesenchymal stem cells are frozen and banked after step (b) or (c). Suitable methods of cryopreserving cells are known in the art and include, for example, incubating the cells in a cryopreservation solution and freezing at least −80° C. Suitable cryopreservation solutions that are known in the art and result in a minimal loss of viability of the cells after a freeze/thaw cycle.

In some examples, the administering of step (d) comprises local delivery of the expanded c-Kit+CD45− mesenchymal stem cells directly into the inflammatory lesions within the gastrointestinal system of the subject. Suitable methods of direct local delivery include, but are not limited to, for example, inter-lesion injection, rectal administration, among others. Cells may also be administered via intravenous injection with means to target areas of intestinal inflammation.

In some embodiments, the disclosure provides methods and composition for treating intestinal inflammation. The methods and compositions provided herein may be combined with any methods or compositions known in the art for the treatment of intestinal inflammation or IBD. Such treatment may include, but is not limited to immunosuppressive drugs, anti-inflammatory drugs, biologics, antibiotics, anti-diarrheal medications, pain relievers, vitamins or supplements, nutritional support or changes, and surgery.

### Compositions

The present disclosure also provides compositions for use in treating gastrointestinal or intestinal inflammations, including, inflammatory lesions. The compositions comprise in vitro culture expanded c-Kit⁺CD45⁻ mesenchymal stem cells capable of decreasing intestinal inflammation. Suitably the compositions comprise at least 80% of cells expressing c-Kit+CD 45−, alternatively at least 85%, alternatively at least 90%, alternatively at least 95%, 96%, 97%, 98%, or 99% c-Kit+CD 45−MCs.

The composition may further comprise a pharmaceutically acceptable carrier. The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch, cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch, cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The compositions described herein may be co-administered with other compositions known in the art for the treatment of intestinal inflammation or IBD. As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term. As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Neonatal Mesenchymal Stem Cells for the Treatment of Ileitis

Neonatal mesenchymal stem cells (nMSCs) are a potent source of cells that possess anti-inflammatory and pro-regenerative characteristics. nMSCs can be harvested from patients then expanded and further banked for future use. nMSCs are also immune privileged and can bypass host immune reactions following delivery. Here, we propose the use of this novel, cellular therapeutic platform to positively modulate the localized inflammatory milieu in a mouse model of Crohn's disease-like ileitis.

### Generation of Neonatal Mesenchymal Stem Cells

C-kit⁺/CD45⁻ nMSCs were isolated from right atrial appendages (RAA) samples using a previously described protocol (Simpson et al, Circulation 2012, Wehman et al, Ann Thoracic Surgery 2017, Sharma et al Circ. Research 2017). Briefly, samples were minced in Ham's F12 medium (Lonza #12-615F) and digested with 1-2 mg/ml of Collagenase type II (Worthington #4177) and incubated on an orbital shaker at 200 rpm on 37° C. for 30-45 min. Following the collagenase treatment, cells were washed twice with growth medium [Ham's F12, Fetal Bovine Serum (10%), recombinant human FGF-basic (10 ng/ml), L-glutathione (0.2 mM), human erythropoietin 250 (5 U/ml)] before being plated. At sub confluency, cells were trypsinized using TrypLE Express (Gibco #12604-013) for 5 min and the cells were collected in Hams-F-12 media. The cell numbers were counted using automated cell counter (Biorad #1450102) and centrifuged at 1000 rpm for 5 min at 4° C. Following centrifugation, the supernatant was discarded, and the cell pellet was resuspended in cryopreservation media (Millipore #C2874) and banked at liquid nitrogen tank or sorted for c-kit⁺ expressing nMSCs cells with Miltenyi microbeads (CD117 MicroBead Kit human #130-091-332) as per the kit instructions. The c-kit⁺/CD45⁻ nMSCs were collected and cultured in growth medium as mentioned above.

### Phenotypic Characterization of Nmscs

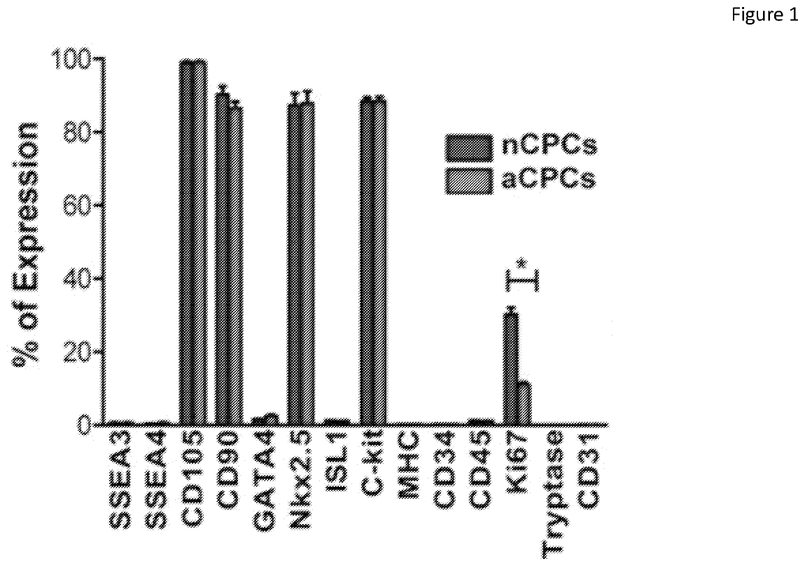

C-kit⁺/CD45⁻ cells derived from neonatal and adult patients at passage 3 were labeled with fluorochrome-conjugated primary antibodies specific for embryonic stem cell markers SSEA4 or SSEA3, mesenchymal stem cell marker CD105 or CD90, cardiac-specific transcription factors NKX2.5, GATA4 and ISL-1, cardiac stem cell marker c-kit, endothelial cell marker CD31, mast cell marker tryptase, hematopoietic cell lineage markers CD45 and CD34. Conjugated isotype antibodies were used as a negative control. The labeled cells were evaluated by flow cytometry with Becton-Dickinson FACS caliber (San Jose, CA) and 25,000 events/sample were collected. At passage 3 (P3), nMSCs and aMSCs had similar cellular morphologies and were negative for expression of hematopoietic markers (CD34 and CD45), endothelial markers (CD31; PECAM-1), mast cell markers (tryptase), certain cardiomyocyte lineage-specific transcription factors (GATA4 and ISL1), and embryonic stem cell markers (SSEA3 and SSEA4). They exhibited equivalent expression of c-kit, mesenchymal stem cell markers (CD105 and CD90) and the cardiomyocyte lineage-specific transcription factor Nkx2.5 (FIG. 1).

### nMSCs Transplantation in Pre-Clinical Mice Model

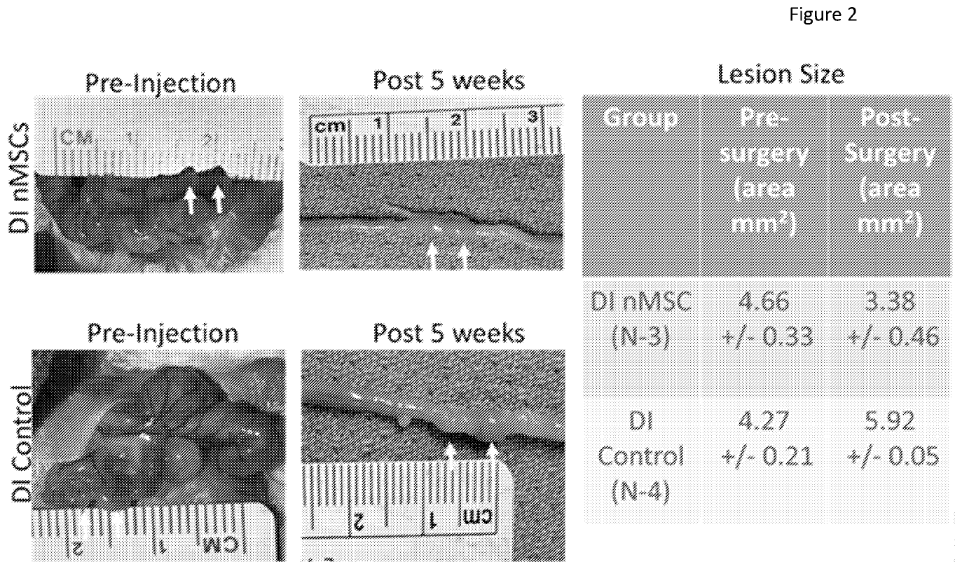

Human neonatal MSCs were administered as previously described (Bury et al, Advanced Therapeutics 2021, Bury et al, Central European Journal of Urology 2014). Briefly, SAMP1 mice (female, 10 weeks old) was injected with nMSCs (20 million/kg in 30 ul) into inflammatory small bowel lesions (n=4 lesions per mouse; n=3 mice in this group) and saline injected mice named as placebo controls. Non-injected mice served as controls. Lesion size pre/post-injections were evaluated (FIG. 2). Mice were euthanized at 15 weeks and individual lesions were collected for tissue specimen processing and staining. Animal studies were approved by the Institutional Animal Care and Use Committee at the center for comparative medicine, Northwestern University, Chicago.

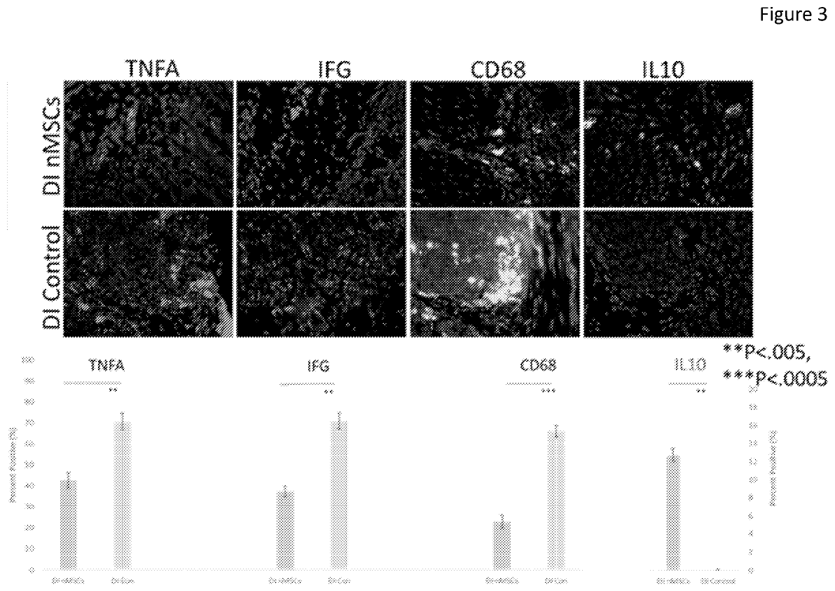

Subsequent tissue sections were single-stained with immunofluorescent antibodies for inflammatory cells CD68 (macrophages), and cytokines TNFα, IFNγ, and IL-10. Quantitative morphometric analyses were performed on stained tissue sections using ImageJ (n=3 images/lesion). Quantified morphometric data represented as percent positive cell or cytokine marker derived from tissue staining experiments with demonstrable decreases in key inflammatory events under DI £ nMSCs vs DI Control conditions. Specifically: TNFα (42.7±3.6 vs 70.6±3.9); IFNγ (37.4±2.5 vs 70.9±3.7) CD68 (23.0±3.1 vs 66.1±2.8). This was accompanied by an increase in anti-inflammatory IL-10 expression (12.8±0.7 vs 0.1±0.01) and a decrease in lesion size (3.38±0.05 mm2 vs 5.92±0.05 mm2), which is a 27.5% decrease in initial lesion size and a 41.9% decrease in size when compared to control group (P<0.05) (FIG. 3).

Chronic intestinal exposure to inflammatory factors exhibited in CD contribute to tissue destruction and impair tissue wound healing. Within the context of our study, we establish that nMSCs can substantially decreases the presence of and down-regulate the expression of pro-inflammatory related cells and cytokines, respectively, in vivo. nMSCs may provide an alternative option for those with CD as treatment also reduces lesion size and promotes wound healing via the upregulation of the pro-regenerative cytokine IL-10.

### Efficacy of Cardiac Progenitor Cells (CPCs) to Reduce Intestinal Inflammation in CD-Like Ileitis

Human CPCs were given intravenously (tail vein; at either 20×10⁶, 10×10⁶, 1×10⁶ CPCs) to 10 week old SAMP mice (100% disease penetrance; n=4 animals/dose). Basal media injected SAMP mice were used a controls (n=3). Ileum and the colon were harvested, immunostained, and quantified 5 weeks post-injection. SAMP mice are an established pre-clinical model for CD and possess striking anatomical and physiological similarities with regards to disease establishment and progression to their human counterparts. These mice are fully immune competent.

### Reduction Skip Lesion Size in the Ileum Following CPC Treatment

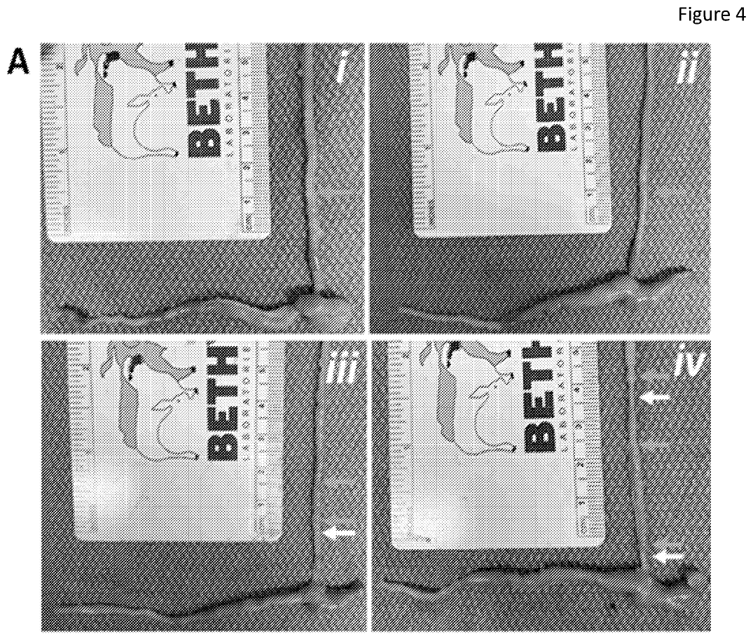

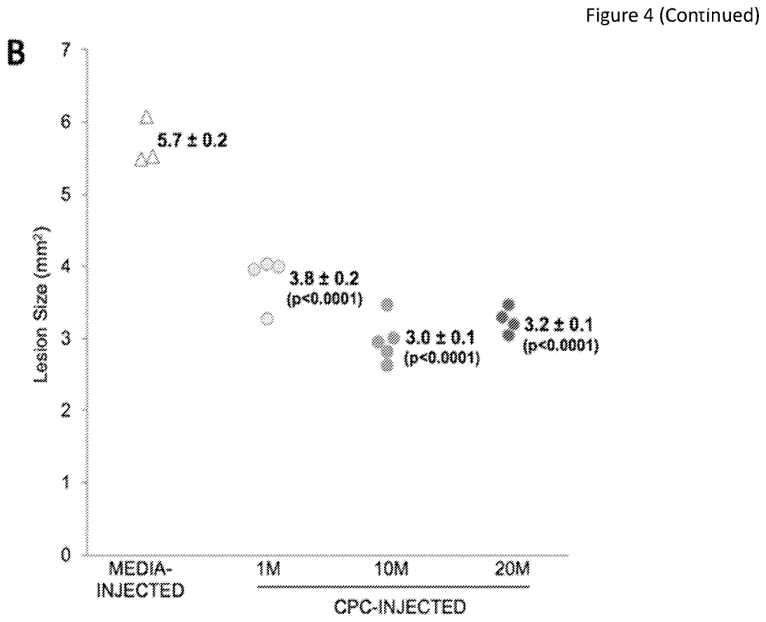

Pro-inflammatory, transmural skip lesions are pathogenic for CD. As the disease progresses, lesion size increases and presents with an internal environment of inflammatory innate and adaptive immune cells and their inflammatory cytokines and chemotactic factors. Left untreated, tissue necrosis ensues. Gross pathology presented in FIG. 4a (i-iv) shows a demonstrable decrease in lesion size and number, 5 weeks post CPC injection. There were no observable gross abnormalities either on the inside (lumen) or outside surfaces of the explanted intestinal tissue from CPC injected animal which was in contrast to media-injected animals that demonstrated increased number of lesions as well as strictures and fissure formation. Images representative of multiple animals. Quantification of the lesion area demonstrate an approximate 48% reduction in lesion size at the midrange CPC dose when comparing CPC-treated to animals untreated animals (FIG. 4b). p<0.0001 was considered statistically significant. There were no observable or measurable negative reactions to CPC injections based upon gross, microscopic, and physiological measures of overall animal disposition or intestinal health.

### Reduction in Ileum-Invading Inflammatory Cells and Cytokines

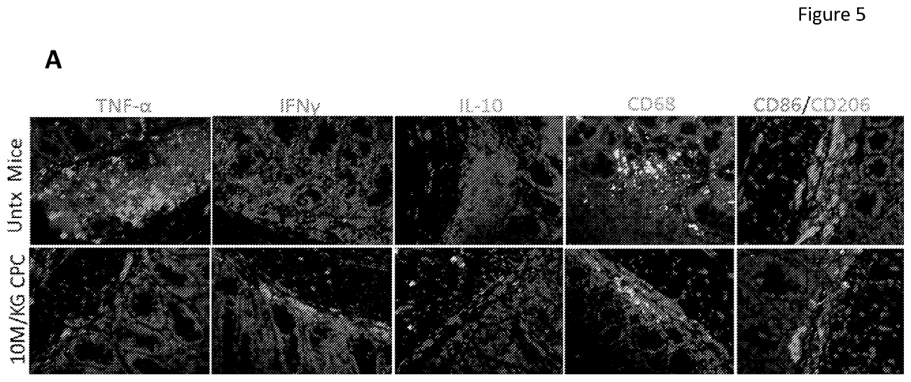

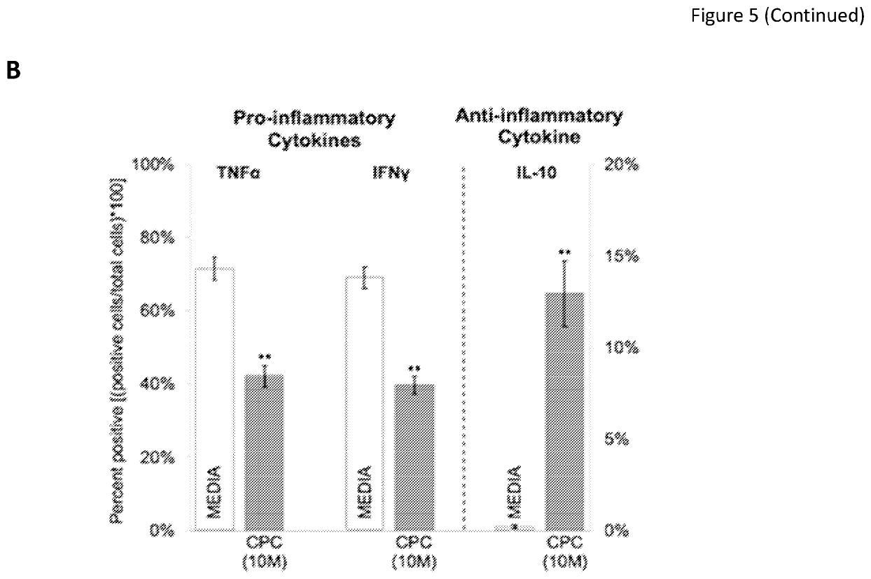

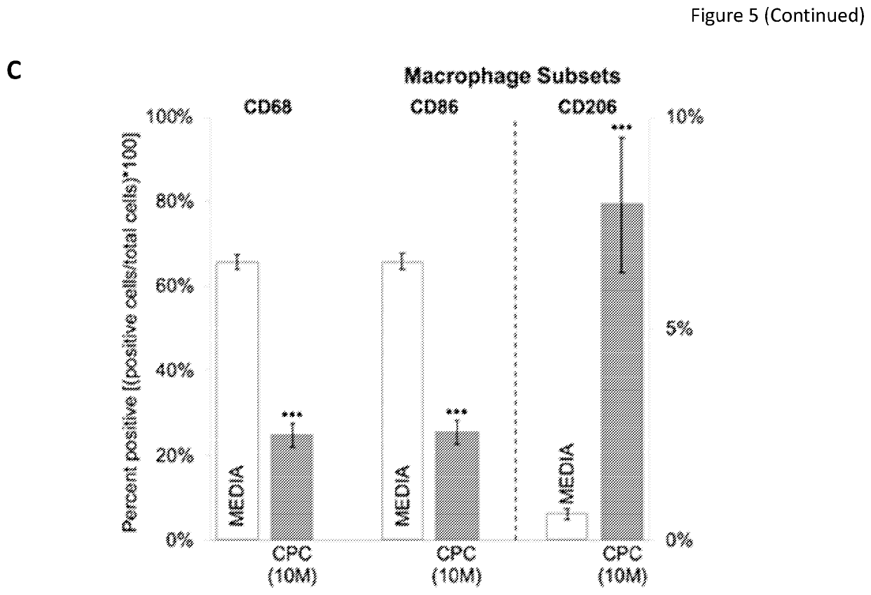

Immunofluorescence (IF) staining of skip lesions revealed a significant decrease in pro-inflammatory cytokines TNFα and IFNγ (FIG. 5) of 10M CPC treated mice. The overexpression of these cytokines is known to exacerbate the inflammatory milieu and lead to tissue destruction. This was accompanied by the downregulation of CD68+ pro-inflammatory macrophages, compared to media control-injected animals. There was a striking up-regulation of the anti-inflammatory, pro-regenerative cytokine IL-10. Tissue IF staining data was quantified and revealed an ~1.80× decrease in TNFα, IFNγ, levels and an ~2.6× decrease in tissue CD68+ M1 macrophages, accompanied by an ~14× increase in anti-inflammatory/pro-regenerative IL-10 levels. Similarly, CD206+ M2 macrophages were significantly upregulated. All data was considered statistically significant. Images representative of multiple animals.

### Intestinal Transit Following Injection of CPCs

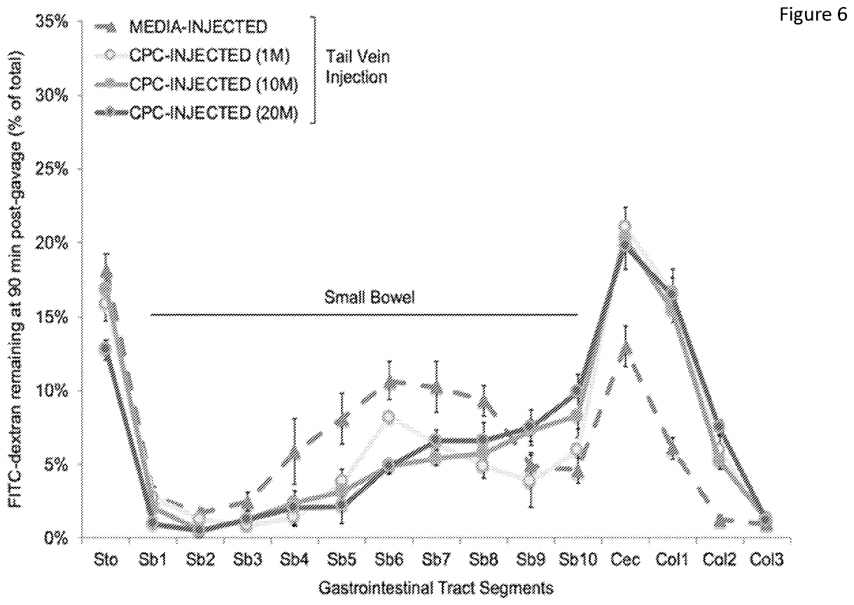

Decreased intestinal transit as a result of tissue fibrosis exhibited in ileitis can lead to weight loss, nutrient malabsorption, prolonged intestinal transit times, and growth perturbations. To assess intestinal transit, CPCs or media were independently provided intravenously to SAMP mice. At 5 weeks post-injection, mice were gavaged with FITC-dextran. Intestinal transit was assessed 90 minutes post-gavage followed by euthanization. For each group, the post-gavage distribution of a FITC-dextran across 15 segments of the gastrointestinal tract was examined (stomach to colon; each segment as percent of total fluorescence). Across the small bowel (segments numbered SB1-SB10; SB1 most proximal and SB10 most distal), marker distribution weighted toward more proximal segments was considered indicative of a slowing of transit. Media-injected SAMP mice demonstrated significant transit delay from SB3 through SB8 compared to CPC treated animals. There was no evidence of diarrhea or bloody stool in mice. (FIG. 6) However, media-injected SAMP mice displayed numerous skip lesions and strictures. (FIG. 4iv) Cecum and Col segments generally accumulate the FITC-dextran which explains the high fluorescent reading.

The geometric center (GC) which is the center of gravity for the distribution of FITC-dextran, was calculated using the formula GC=Σ(% of total fluorescent signal per segment*segment number)/100. By multiplying percentage of fluorescence of each segment with its segment number, a weighted mean of the distribution of marker within the intestine was obtained. In the media-injected group, SB mean fluorescence peaked at segments SB6-SB7 (10.7±1.3, 10.2±1.8), with levels >5% for SB4-SB8, resulting in a mean GC of 7.2±0.3. SB mean fluorescence for the CPC-injected (1M) group peaked at SB6 (8.2±0.3), with levels >5% for fewer SB segments in the region (SB6-SB7). For CPC-injected (10M) and (20M) groups, SB mean fluorescence peaked more distally, at segment SB10 [8.3±1.4 (10M); 9.9±1.2 (20M)]. Mean GC values for all CPC-injected groups were significantly greater than the media-injected mean GC [media-injected 7.2±0.3 vs CPC-injected (1M) 8.9±0.2(p<0.001), CPC-injected (10M) 8.9±0.2 (p<0.001), CPC-injected (20M) 9.5±0.1 (p<0.0001)].

### Example 2

Reference is made to the manuscript: Gunasekaran et al., Multipotent Human Neonatal Cardiac-Derived Mesenchymal Stem Cells Modulate Ileitis in vivo,” the content of which is incorporated herein by reference in its entirety.

### Results

### nMSCs Injection Reduces Ileal Lesions and Alleviates Tissue Pathogenesis

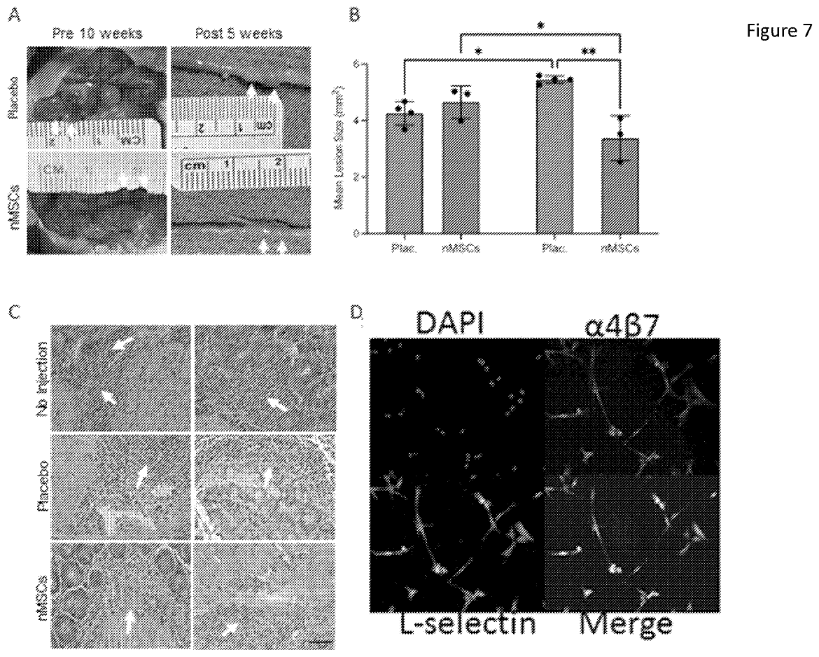

We analyzed the effect of nMSCs to attenuate lesion progression by comparing pre-treatment SAMP mice at 10 weeks of age to animals at 15 weeks of age (5 weeks post-treatment) via evaluation of lesion size and infiltration of inflammatory immune cells. The skip lesions in the ileal tissue were measured prior to the injection of nMSCs and placebo (saline-injected SAMP mice) control in 10 week old mice (FIG. 7a). At pre-injection, ileal lesion sizes (5 lesions/animal represented as mm²) were comparable across all the conditions and between the groups (placebo 4.2±0.4; nMSCs 4.6±0.5; p=0.734) (FIG. 7a). Macroscopic evaluation at 5 weeks post-treatment showed significant reduction in ileitis as assessed by lesion size in nMSCs treated mice compared to the placebo control (placebo 5.4±0.1 vs nMSCs 3.3±0.7; p=0.001) (FIGS. 7a, b). Histological staining on ileal lesions showed that nMSCs treatment markedly reduced infiltration of inflammatory cells and increased epithelial cells adjacent to the lesions compared to the placebo control (FIG. 7c).

### nMSCs Modulate Macrophage Sub-Types in Ileal Lesions

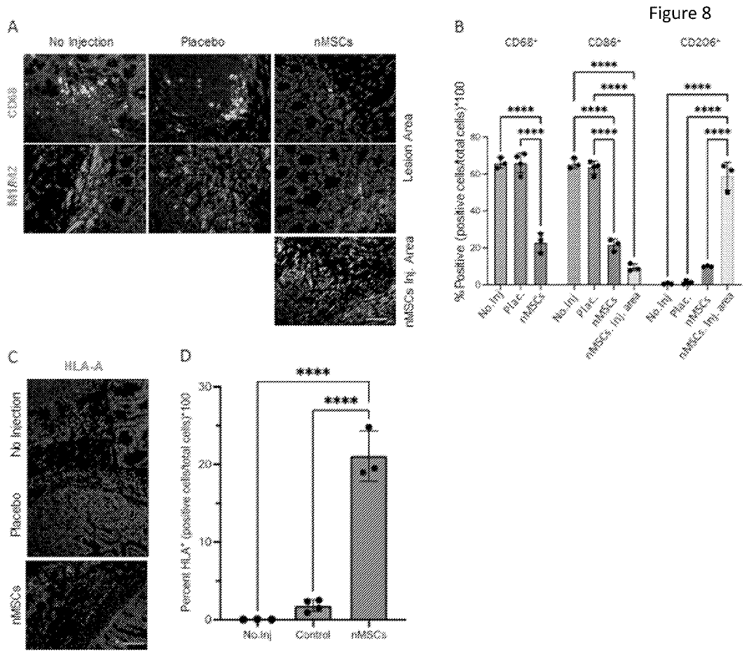

Histological examination of ileal tissue showed a statistically significant reduction of CD68⁺Mφ within lesions following nMSC treatment compared to placebo control at post 5-week treatment [NI (no injection) 66.0±2.9, placebo 66.1±5.6, nMSCs 23.0±5.3; NI vs nMSCs p<0.0001, placebo vs nMSCs p<0.0001, NI vs placebo <0.9999)] (FIGS. 8a, b). Similarly, the percentage of inflammatory M1 Mφ (CD86⁺M1 Mφ) was reduced in nMSCs injected mice compared to no injection and placebo control (NI 65.7 2.8, placebo 63.5±3.5, nMSCs 21.6±3.3; NI vs nMSCs p<0.0001, placebo vs nMSCs p<<0.0001, NI vs placebo <0.9985). In contrast, the anti-inflammatory/pro-reparative M2 Mφ (CD206⁻ M2 Mφ) were significantly increased in mice injected with nMSCs compared to placebo (NI 0.7±0.4, placebo 1.3±0.8, nMSCs 10.0±0.4; NI vs nMSCs p<0.0489, placebo vs nMSCs p<<0.0487, NI vs placebo<0.9963). Interestingly, we observed significant reduction of M1Mφ (nMSCs inj. area 9.3±1.9; NI vs nMSCs inj. area p<0.0001, placebo vs nMSCs inj. area p<<0.0001, NI vs placebo <0.9995, nMSCs vs nMSCs inj. area p<0.0205) and increase of M2Mφ (nMSCs inj. area 58.8±7.7; NI vs nMSCs inj. area p<0.0001, placebo vs nMSCs inj. area p<<0.0001, NI vs placebo <0.9999, nMSCs vs nMSCs inj. area p<0.0001) in nMSCs injection lesion site which indicate that nMSCs modulate inflammatory M1 Mφ into anti-inflammatory M2 Mφ to mitigate inflammation in the SAMP mice. The retention of nMSCs in the lesion tissue were identified using human leukocyte antigen-A (HLA-A) (NI 0.03±0.01, placebo 1.8±0.7, nMSCs 21.1±3.2; NI vs nMSCs p<0.0001, placebo vs nMSCs p<<0.0001, NI vs placebo <0.4358). Histological examination of ileal skip lesions showed increased percentage of nMSCs in post-treatment at 15 weeks suggesting that increased nMSCs retention rate might contribute to the inflammation resolution and intestinal epithelial regeneration (FIGS. 8c, d).

### nMSCs Regulate Inflammatory Cytokines and Retain in the Lesion Tissue at Post 5 Weeks Treatment

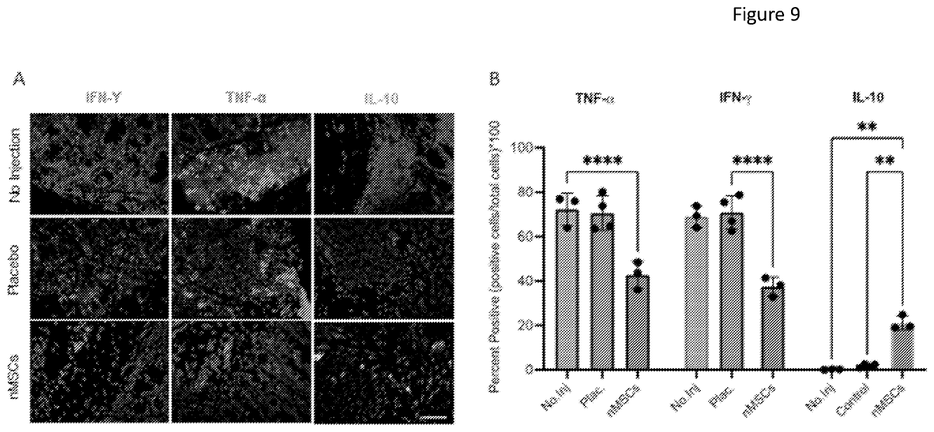

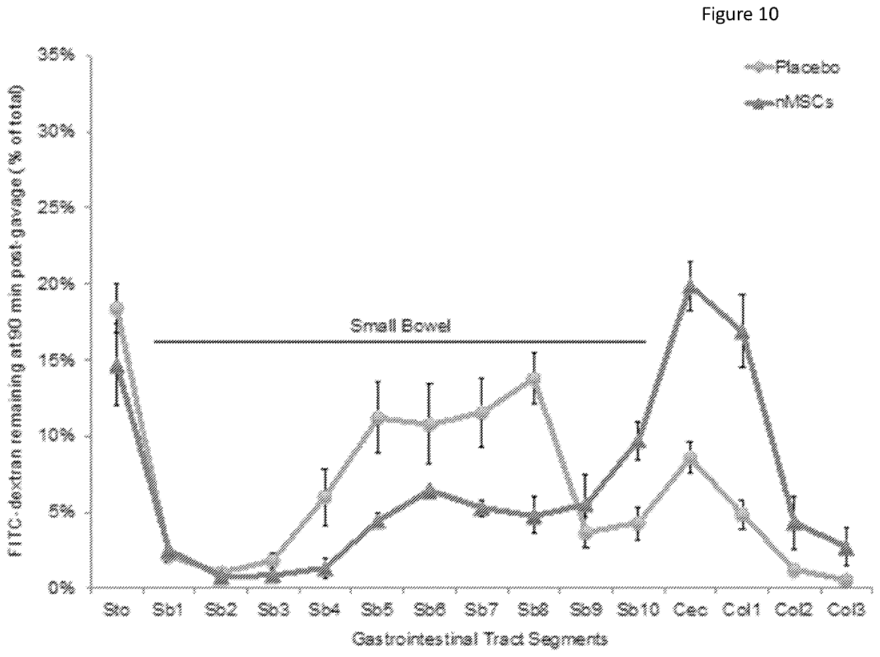

To corroborate tissue immune cell modulation by nMSCs, we evaluated the levels of pro and anti-inflammatory cytokines in the lesion tissue by immunofluorescence analysis. The lesion-specific pro-and anti-inflammatory cytokine analyses revealed that inflammatory cytokines TNF-α and IFN-y were significantly reduced following nMSC injection compared to placebo and non-injection (NI) controls at 5 weeks post-treatment (TNF-α: NI 72.3±7.3, placebo 70.6±7.8, nMSCs 42.7±6.2; NI vs nMSCs p<0.0001, placebo vs nMSCs p<0.0001, NI vs placebo p<0.9999, IFN-y: (NI 69.1±4.9, placebo 71.0±7.5, nMSCs 37.4±4.3; NI vs nMSCs p<0.0001, placebo vs nMSCs p<<0.0001, NI vs placebo p<0.9999) (FIGS. 9a, b). In contrast, upregulation of anti-inflammatory cytokine IL-10 was observed following nMSC treatment compared to non-injection and placebo control (NI 0.2±0.2, placebo 0.1±0.0, nMSCs 12.8±1.2; NI vs nMSCs p<0.0001, placebo vs nMSCs p<0.0001, NI vs placebo p<0.9999). Data indicate that nMSC administration can modulate inflammatory immune cells into the regulatory or anti-inflammatory phenotype resulting in increased anti-inflammatory cytokine IL-10 release in the lesions of the ileitis model. Therapeutic efficacy of nMSCs on small bowels and intestinal pathology To further investigate GI transit and a segment gross pathology score (SGPS) following nMSCs treatment, we performed an established fluorescein isothiocyanate (FITC) dextran motility assays.³⁰ The GI tract was divided into 15 segments (S1 to S15; sections 1-10 are small bowel and 11-15 are cecum and large intestine) and the percent total fluorescence in each segment was examined to create a SGPS. The appearance of increased proximal distribution of FITC-21 dextran is indicative of delayed GI transit and enhanced gross diseases pathology between SB6 to SB10. The SAMP mice injected with nMSCs showed reduced distribution of FITC dextran from segment SB6 up to distal direction suggesting reduced ileitis and histology scores (reduced lesions and strictures) (FIG. 10). nMSCs: SB6 6.4±0.1, SB7 5.3±0.9, SB8 4.7±1.2, SB9 5.5±2.0, SB10 9.7±1.2. Conversely, the placebo control showed the presence of significantly increased proximal distributions peaks indicating delayed GI transit due to severe lesions and ileitis. Placebo control: SB6 9.9±2.6, SB7 11.8±2.2, SB8 14.8±1.7, SB9 3.3±1.1, SB10 4.2 1.1. The occurrence of heightened peak in the placebo groups is suggesting that the development of increased strictures in the small bowel segments and pathology score. Conclusively, the direct injection of nMSCs mitigated small bowel motility dysfunction and intestinal obstruction compared to placebo controls suggesting that, nMSCs injection promote the regeneration of epithelial cells to improve the GI transit in SAMP mice.

### Methods

### SAMP1/YitFcsJ Animal Model

The SAMP1/YitFcsJ (“SAMP”; Jackson Laboratories) mouse model exhibits CD-like ileitis and mimics aspects of human CD and thus can be extrapolated to the human condition. It is an accepted pre-clinical model of CD-like ileitis.³⁰ Gross observations/clinical symptoms of SAMP mice include luminal blockage, skin crypt elongation, lesions, intestinal perforations, villus loss, transluminal inflammation, fissure and fistula formation, impaired nutritional uptake, and delayed small bowel transit at the age of 10 weeks. At the microscopic level, epithelial hyperplasia of Paneth and goblet cells becomes evident along with disproportionate villus/crypt ratios within the ileum. Mice approach 100% penetrance at nearly 10 weeks of age. Protracted expression of TNF-α and IFN-y is also evident at early stages of life and gradually accrues overtime and disease progression. All animal procedures were performed under the oversight and guidance of the Northwestern University Institutional Animal Care and Use Committee (IACUC) through an approved animal protocol (#IS00018168). SAMP mice were divided into three groups: i) no-injection, ii) placebo (saline injection), and iii) nMSC injection group.

Neonatal mesenchymal stem cells (nMSC) preparation Human cardiac derived nMSCs were isolated from right atrial appendages biopsies collected from congenital heart diseases patients as described earlier.^{29,48, 55,59} Biopsy samples were collected after informed consent and approved by the Northwestern University/Lurie Children's Hospital Institutional Review Board (IRB; #2006-12796). nMSCs were characterized for the stem cell markers (CD105 or CD90), cardiac stem cell marker c-kit, endothelial cell marker CD31, cardiac-specific transcription factors NKX2.5, GATA4 and ISL-1, hematopoietic cell lineage markers CD45 and CD34, mast cell marker tryptase, and the cell proliferation marker Ki67.^{29, 55,59} c-kit⁺/CD45⁻ nMSCs were isolated from right atrial appendages (RAA) samples using a previously described protocol.^{29, 55,59} Briefly, samples were minced in Ham's F 12 media (Lonza, Inc.) and digested with 1-2 mg/ml of collagenase type II (Worthington, Inc.) and incubated in an orbital shaker at 200 rpm, 37° C. for 30-45 min. Following the collagenase treatment, cells were washed twice with growth media [Ham's F12, Fetal Bovine Serum (10%), recombinant human FGF-basic (10 ng/ml), L-glutathione (0.2 mM), human erythropoietin (5 U/ml)] before being plated. At sub-confluency, cells were trypsinized and sorted for c-kit+expressing nMSCs cells with Miltenyi microbeads (CD117 MicroBead Kit Human #130-091-332) as per manufacturer's instructions. The c-kit/CD45 nMSCs were collected and cultured in growth media as described above.

### Small Intestinal Nmsc Lesion Direct Injection

SAMP mice (~15-25 grams; 10 weeks of age; female) were provided an ad libitum liquid diet (Jevity; 1 cal Isotonic Nutrition and Fiber) for 24 hour prior to surgery and nMSC small intestinal injections. Immediately prior to surgery, 2% isoflurane with O 2 anesthesia was administered and a midline abdominal incision (1.0-1.5 cm) was made at the linea alba to gain access to the abdominal cavity and to identify the small intestines. Following skip lesion identification on the small intestine, animals were either injected with nMSCs (20 million/kg in 30 μl of media into the skip lesions (n=5 lesions/mouse; n=3 mice), or injected with saline (30 μl; termed as placebo controls; n=5/mouse, n=4 mice) by the handler utilizing a 27 gauge needle. Non-injected mice served as unmanipulated controls (n=3). The injected areas were marked with India ink and then identified using measurements from the cecum. Organs and intestines were relocated to their original anatomical positions following the procedure and the intestines were gently rinsed with sterile saline prior to return to the peritoneal cavity. The abdomen was closed in 2 layers that included the muscle and skin with 4.0 PDS, and 4.0 Ethilon used for skin closure. Following recovery, animals were placed back into their respective cages with non-edible bedding. They were provided ad lib water and liquid diet for the first 7 post-operative days following recovery. Animals were returned to regular chow 7 days post-op and monitored on a regular basis.

### Tissue Specimen Processing and Histological Staining

As described,³⁰ skip lesions were identified based upon India ink marking and removed from animals following euthanasia at 5 weeks post-injection. Lesion size was determined using Calipers (Mitutoyo Absolute Digital Caliper) were utilized to measure lesion size based upon the length and width of the lesions. Individual lesions were fixed in a 10% buffered formalin phosphate, dehydrated with graduated ethanol solutions, and finally embedded in paraffin wax molds. Molds were then sectioned onto glass slides at a 5-7 μm thickness and allowed to air dry. Slides were then stained with an established H&E staining protocol (or used for immunofluorescence studies) and a coverslip was attached using Permaslip (Alban Scientific Inc.).³⁰

### Immunofluorescence Staining

The aforementioned prepared slides were deparaffinized, dehydrated with ethanol and rehydrated in diH 20 before being placed in boiling antigen retrieval buffer (0.01 M citrate, pH 6.0, 0.05% Tween-20) for 15 minutes, and then allowed to cool to room temperature for 30 minutes. Following cooling, tissue sections were circled with a hydrophobic pen and blocked with bovine serum albumin (BSA-5 mg/ml) (Sigma-Aldrich, MO, USA) for 15 minutes. Intestinal tissue lesions collected and embedded in paraffin were cut into 5 μm-thick sections and stained with individual primary antibodies targeting IL-10, IFN-y, CD68, CD86, CD206, TNF-α, HLA-A [IL-10 at 5 μg/mL, IFN-y at 10 ug/ml, CD68 at 10 μg/mL, CD86 at 5 μg/ml, CD206 at 5 ug/ml, TNF-α at 9 μg/mL, HLA-A at 10 μg/ml); secondary antibodies ranged from of 2-4g/ml] in 1% BSA and incubated for 1 hr at room temperature. Slides were washed 3× with 1× PBS and then incubated with respective secondary antibodies for 30 min at room temperature. Following incubation, slides were washed 3× with PBS. The cell nuclei were stained with DAPI (4′,6-diamidino-2-phenylindole) and the slides were visualized under a Nikon Eclipse 50i microscope and Spot advanced Imaging Software (Diagnostic Instruments, MI, USA). Individual primary antibodies were obtained from Abcam (MA, USA). Secondary antibodies: Goat-Anti-Rabbit 555, Goat-Anti-Mouse 488 diluted 1:400 were obtained from Invitrogen Corp., CA, USA.

### Immunofluorescence Quantification

An IF quantification protocol was adapted from our previous study.³⁰ Quantification of fluorescently stained tissue sections was accomplished using a Nikon Eclipse 50i microscope and Spot Advanced Imaging Software (Diagnostic Instruments, MI, USA). Of the treated or non-treated lesions per animal, 3 images per lesion were taken (a total 12 total images per animal; n=3 animals/experimental group). The number of percent positive inflammatory cells was determined by manually counting fluorescently stained cells using the ImageJ cell counter plugin function software (National Institutes of Health). The total number of cells in each image was determined by opening the DAPI channel alone and converting the image threshold to only highlight DAPI+ cells. To eliminate stacked cells, the watershed function of ImageJ was used. This was followed by the use of the analyze particle tool to determine total cell count. The size was adjusted to 180 pixels-500 and circularity set from 0.0-1.0.³⁰ Intestinal Motility Prior to motility assay, animals were fasted for 12 hours as previously described in detail.³⁰ Mice received an oral gavage of 100 mg/ml FITC-dextran (in Kreb's solution; 44 mg/100g body weight; 70,000 MW, Sigma Aldrich). At 5 weeks post-treatment and after 90 minutes post FITC-dextran treatment, animals were humanely euthanized, and the GI tract was carefully removed and included the stomach through the colon. The small intestine was divided into 10 segments (SB) and the colon (Col) was divided into 3 segments. Each segment was flushed with 1 ml of Kreb's solution and the subsequent wash solution was collected. Following collection, samples were centrifuged, the supernatant was collected and read on a plate reader at 488nm/519nm. The total accumulation of FITC-dextran was determined from individual tissue segments and quantified.

Statistical Analysis The statistical analysis was performed using GraphPad Prism 9 (GraphPad Software, La Jolla, CA, USA). Comparative analysis for cytokines (IFN-y, TNF-α and IL-10 ), immune cells (CD68⁺, CD86⁺, CD206⁺) and HLA-A detection among no-injection, placebo and nMSCs were achieved by one-way analysis of variance with Tukey's multiple comparisons. The intestinal healing following nMSCs injection and placebo control was analyzed by Mann-Whitney U test. Small bowel transit delay between nMSCs and placebo control was calculated using one-way ANOVA with Dunnett's post hoc test. *p<0.05, **p<0.01, ***p<0.001, ****p<0.0001 was considered statistically significant.

### Discussion

Crohn's disease is comprised of chronic inflammation in the proximal, distal small intestine and in the mucosa of the colon resulting in destructive intestinal epithelium due to dysregulated immune cells activation. 31 Immunotherapy (anti-TNFα; infliximab, anti-integrins, Vedolizumab, anti-IFN-y; Fontolizumab) has been showed as effective therapy for CD and has greatly improved quality of life of CD patients in various clinical trials.32-35 Recent studies have also showed that steroids and thiopurine can modulate the inflammation and promote mucosal healing in the chemically induced murine colitis model.11 However, the complications of the aforementioned therapies include immune reaction, development of skin lesions, peri-operative morbidity, post-management infections and increased risk for malignancies, which warrants safe and efficient therapy for CD.11,35,36 Also, the non-specific immune suppression and its side effects warrant efficient therapy not only to suppress systemic inflammation but to also promote epithelial wound healing applicable animal models that will advance treatment option for the millions afflicted with CD.

Stem cells have therapeutic effects in treating various immune related disorders due to their immune modulatory and pro-regenerative potential.37-40 MSCs are non-hematopoietic, self-renewing, hypoimmunogenic stem cells and have been shown to attenuate inflammation in chemically induced experimental mouse and rat colitis models^{5,41} MSCs including adipose derived MSCs (AD-MSCs)^{13,42} mbilical cord derived MSCs (UC-MSCs)¹⁵ dental follicle derived MSCs (DF-MSCs)⁴³ and bone marrow derived MSCs (BM-MSCs).^{5,6} The aforementioned cell types have been shown to possess therapeutic efficacy in mitigating inflammation in experimental colitis models. A recent study identified that a single dose of intraperitoneal administered AD-MSCs attenuated intestinal inflammation and restored epithelial barrier integrity in the DSS induced experimental colitis mice model.⁴⁴ Similarly, a study by Gonzalez et al also demonstrated that AD-MSC injections markedly ameliorated clinical and histopathologic severity of colitis and inflammation in TNBS induced colitis in murine models.¹⁶ Although, MSCs have therapeutic potential in the pre-clinical experimental colitis model, the efficacy of MSCs in treating IBD patients in clinical trials remains modest. Also, the chemical induced experimental model is not an ideal model to mimic pathology observed in CD patients. Therefore, in this study, we employed a mouse model that develops CD-like ileitis that resembles intestinal lesions of CD patients in clinical settings.

The immune modulation and wound healing potential of MSCs is highly influenced by the inflammatory milieu of injured tissues.⁴⁵ MSCs isolated from adult donors have reduced proliferation rates and increased immune rejections and thus moderate efficacy in treating tissue injuries.⁴⁶ Similarly, our recent findings demonstrated that the therapeutic efficacy of MSCs is highly influenced by the chronological age of the donors (29). In this regard, the adult MSCs administration showed poor engraftment, low survival, limited paracrine effect and reduced cardiac function outcome in rat MI model.²⁹ Studies have identified that the tissue infiltration of inflammatory immune cells and accumulation of inflammatory cytokines increases hypoxic and apoptotic stimuli that may induce injected MSCs clearance.^{47,48} Recent findings from our group demonstrated that cardiac derived neonatal MSCs (nMSCs) showed superior cardiac functional outcome compared to adult derived MSCs (aMSCs) due to its increased retention and their sustained paracrine secretion in the rat MI model.^{29,49} In this study, we investigated the therapeutic efficacy of nMSCs to protect intestinal epithelium by modulating immune cells and inflammation in the SAMP mice. Consistent with previous findings on MSCs as cellular therapy in the experimental colitis mouse model, histological examination of colon tissue showed that a single dose of direct injection of nMSCs in the ileitis lesions attenuated the transmural inflammation and promoted epithelial cells regeneration in the ileum compared to no-injection and placebo controls in the CD-like ileitis mouse model.

Macrophages (Mφ) are a known driver of inflammation in the GI tract in CD patients. Pre-clinical studies on experimental colitis in the Mφ deficient mice model confirms that Mφ play a key role in promoting intestinal lesions and obstructions.^{50,51} Substantial evidence from various findings revealed that MSCs promote pro-inflammatory M1 Mφ polarization towards anti-inflammatory M2 Mφ.⁵²⁻⁵⁵ To define innate immune modulation potential of nMSCs, we investigated the level of inflammatory M1 Mφ (CD86⁺) and pro-regenerative anti-inflammatory M2 Mφ (CD206⁺) in SAMP mice. Intra-lesional administration of nMSCs modulated inflammation by increasing M2 Mφ resulting in reduced inflammatory M1 Mφ in the ileal skip lesions. Significant increases in anti-inflammatory cytokine IL-10 following nMSCs administration suggests that nMSCs modulate inflammatory MI Mφ into pro-reparative M2 Mφ and the expansion of T-regulatory cells phenotypes resulting in the attenuation of inflammation and regeneration of intestinal epithelial cells.

MSCs polarize inflammatory M1 Mφ into anti-inflammatory M2 Mφ to release anti-inflammatory cytokine IL-10 in order to resolve the inflammation.52-55 Intraperitoneal injections of UC-MSCs increased the level of IL-10 and infiltration of regulatory T cells (T-regs) cells to modulate the inflammation in the DSS and TNBS induced colitis mice model.¹⁵ Similarly, systemic infusion of AD-MSCs attenuated the inflammation by increased IL-10 releasing M2 Mφ and T-regs to suppress Thl effector cells responses in vitro and in vivo.16 Consistent with aforementioned findings, in this study, we have identified significant increase in IL-10 levels in the lesions administered with nMSCs which confirms that nMSCs increases IL-10 producing M2 Mφ and T-regs in the inflammatory milieu. We next examined the half-life of injected nMSCs in the lesion site by targeting HLA. Histological analysis for nMSCs retention revealed that nMSCs can be detected even after 5 weeks of injection which confirms that nMSCs are immunologically tolerant against host immune responses. Interestingly, we found significantly increased M2 Mφ in the nMSCs injected area suggesting that nMSCs modulate inflammatory M 1Mφ into anti-inflammatory M2 Mφ to attenuate the inflammation in ileitis lesions. These results indicate that the intralesional administration of nMSCs are well tolerated against host immune rejection and thus nMSCs can be an ideal stem cell type for a future clinical application to treat CD patients.

Dysregulated immune cells activation releases increased inflammatory cytokines TNF-α and IFN-γ which exacerbates the inflammatory conditions in CD patients.¹⁴ Several studies have identified that the accumulation of inflammatory cytokines including TNF-α and IFN-γ increases intestinal small bowel tissue inflammation and iliac epithelial cells degenerations.^{4,17,42} Direct injection of UC-MSCs attenuated inflammation by decreasing inflammatory cytokines, TNF-α and IFN-γ, in the experimental colitis model.¹⁵ Additionally, UC-MSCs improved macroscopic, histological scores and reduced symptoms associated with colitis. Similarly, AD-MSCs injection reduced the expression of TNF-α and IFN-γ in the colitis model in vivo.16 Herein, we investigated the immune modulatory effect of nMSCs by assessing the presence of inflammatory cytokines associated with CD like ileitis. Histological evaluation of lesion tissue showed that nMSCs administration significantly reduced pro-inflammatory mediators TNF-α and IFN-γ suggesting that nMSCs attenuate inflammatory cytokine producing cells such as macrophages, dendritic cells and cytotoxic T cells. Our results are consistent with previous reports showing the significant reduction in inflammatory cytokines TNF-α and IFN-γ in the mice model of ileitis and their immune modulation potential makes nMSCs a putative stem cell that may be used to treat CD in clinical patients.

FITC-dextran distribution assay was employed to assess the GI transit delay and segment pathology score that are indicative of inflammatory lesions and colon strictures.³⁰ Small bowel GI transit and segment gross pathology analyzed by the FITC-dextran distribution assay revealed that nMSCs injection showed significantly improved GI transit and reduced segment pathology in the ileitis mice. We speculate that these findings suggest that the injected nMSCs promoted the wound healing of epithelial layers and submucosa by vasculogenesis and differentiation of nMSCs into epithelial cells which leads to the improvement in intestinal physiology. Our results not only demonstrated the inflammation resolution by nMSCs, but also showed that the administration of nMSCs promote mucosal healing through their wound healing capabilities in vivo.

Our results demonstrate significant improvement with regards to intestinal physiology and short-term resolution of inflammation. The direct lesion inject of nMSCs may not be ideal in a patient setting where open surgery would be required for the pinpoint delivery of nMSCs to skip lesions. Surgery in this setting is typically reserved to resect irreversibly damaged tissue. As an alternative strategy, nMSCs could potentially be delivered via endoscopy-guided approached to areas of inflammation without the invasiveness of an open surgery. Alternatively, a non-invasive delivery platform of the nMSCs could bypass this issue. MAdCAM-1 is the ligand for α4β7 integrin as well as L-selectin (CD62L). MAdCAM-1 is significantly upregulated in intestinal high endothelial venules (HEVs) during ileitis.⁵⁶⁻⁵⁸ Our preliminary data demonstrate that our nMSCs express α4β7 integrin and L-selectin (FIG. 1d). The blockage of in vivo α4β7 integrin/MAdCAM-1 or L-selectin/MAdCAM-1 interactions through intravenous nMSC delivery may significantly reduce and subsequently prevent ileitis. Delivery can also prevent gut-tropic inflammatory α4β7+CD4 T-cells and other L-selectin+leukocytes from exacerbating localized inflammation as nMSCs will compete with adaptive immune cells for MAdCAM-1 binding. Thus, nMSCs can be used to directly target MAdCAM-1 expressed on HEVs during ileitis via a non-invasive, intravenous injection. Finally, our investigation demonstrated that nMSCs promote epithelial cell layer reformation in the ileum. However, whether nMSCs could be able to differentiate into epithelial cells and relevant colonic cells in the SAMP model is still unclear. nMSCs may be a feasible alternative to current cellular therapeutics used to treat CD.

Within the context of this study, we have demonstrated for the first time that direct injection of human cardiac derived nMSCs could alleviate systemic inflammation and recover intestinal physiology in a murine model of CD-like ileitis. The underlying possible mechanism is that the nMSCs secretome including paracrine factors and exosomes might modulate the inflammatory immune cells into pro-reparative anti-inflammatory immune cells. Similarly, paracrine-released molecules by nMSCs may serve as the basis to promote intestinal physiology. Our data demonstrate that nMSC therapy attenuated the inflammation and induced wound healing in the intestinal tissues that can be taken into consideration to treat CD patients in the clinical settings.

### REFERENCES

1. Molodecky N A, Soon I S, Rabi D M, Ghali W A, Ferris M, Chernoff G, Benchimol E I, Panaccione R, Ghosh S, Barkema H W, Kaplan G G. Increasing incidence and prevalence of the inflammatory bowel diseases with time, based on systematic review. Gastroenterology. 2012; 142(1):46-54.e42.
2. Hugot JP, Chamaillard M, Zouali H, Lesage S, Cézard JP, Belaiche J, Almer S, Tysk C, O'Morain C A, Gassull M, Binder V, Finkel Y, Cortot A, Modigliani R, Laurent-Puig P, Gower-Rousseau C, Macry J, Colombel J F, Sahbatou M, Thomas G. Association of NOD2 leucine-rich repeat variants with susceptibility to Crohn's disease. Nature. 2001; 411(6837):599-603.
3. Ogura Y, Bonen D K, Inohara N, Nicolae D L, Chen F F, Ramos R, Britton H, Moran T, Karaliuskas R, Duerr R H, Achkar J P, Brant S R, Bayless T M, Kirschner B S, Hanauer S B, Nuñez G, Cho J H. A frameshift mutation in NOD2 associated with susceptibility to Crohn's disease. Nature. 2001; 411(6837):603-6.
4. Molendijk I, Barnhoorn M C, de Jonge-Muller E S, Mieremet-Ooms M A, van der Reijden J J, van der Helm D, Hommes D W, van der Meulen-de Jong A E, Verspaget H W. Intraluminal injection of mesenchymal stromal cells in spheroids attenuates experimental colitis. J Crohns Colitis. 2016; 10(8):953-64.
5. He X W, He X S, Lian L, Wu X J, Lan P. Systemic infusion of bone marrow-derived mesenchymal stem cells for treatment of experimental colitis in mice. Dig Dis Sci. 2012; 57(12):3136-44.
6. Ciccocioppo R, Bernardo M E, Sgarella A, Maccario R, Avanzini M A, Ubezio C, Minelli A, Alvisi C, Vanoli A, Calliada F, Dionigi P, Perotti C, Locatelli F, Corazza GR. Autologous bone marrow-derived mesenchymal stromal cells in the treatment of fistulising Crohn's disease. Gut. 2011; 60(6):788-98.
7. Shi H Y, Ng S C. The state of the art on treatment of Crohn's disease. J Gastroenterol. 2018; 53(9):989-98.
8. Pagnini C, Pizarro T T, Cominelli F. Novel pharmacological therapy in inflammatory bowel diseases: beyond anti-tumor necrosis factor. Front Pharmacol. 2019; 10:671.
9. Adegbola S O, Sahnan K, Warusavitarne J, Hart A, Tozer P. Anti-TNF therapy in Crohn's disease. Int J Mol Sci. 2018; 19(8).
10. Safarpour A R, Hosseini S V, Mehrabani D. Epidemiology of inflammatory bowel diseases in Iran and Asia; a mini review. Iran J Med Sci. 2013; 38(2 Suppl): 140-9.
11. Neurath M F. Current and emerging therapeutic targets for IBD. Nat Rev Gastroenterol Hepatol. 2017; 14(5):269-78.
12. Chao K, Zhang S, Qiu Y, Chen X, Zhang X, Cai C, Peng Y, Mao R, Pevsner-Fischer M, Ben-Horin S, Elinav E, Zeng Z, Chen B, He Y, Xiang AP, Chen M. Human umbilical cord-derived mesenchymal stem cells protect against experimental colitis via CD5(+) B regulatory cells. Stem Cell Res Ther. 2016; 7(1):109.
13. Gao J G, Yu M S, Zhang M M, Gu X W, Ren Y, Zhou X X, Chen D, Yan T L, Li Y M, Jin X. Adipose-derived mesenchymal stem cells alleviate TNBS-induced colitis in rats by influencing intestinal epithelial cell regeneration, Wnt signaling, and T cell immunity. World J Gastroenterol. 2020; 26(26):3750-66.
14. Wang G, Joel M D M, Yuan J, Wang J, Cai X, Ocansey D K W, Yan Y, Qian H, Zhang X, Xu W, Mao F. Human umbilical cord mesenchymal stem cells alleviate inflammatory bowel disease by inhibiting ERK phosphorylation in neutrophils. Inflammopharmacology. 2020; 28(2):603-16.
15. Kim H S, Shin T H, Lee B C, Yu K R, Seo Y, Lee S, Seo M S, Hong I S, Choi S W, Seo K W, Núñez G, Park J H, Kang K S. Human umbilical cord blood mesenchymal stem cells reduce colitis in mice by activating NOD 2 signaling to COX 2. Gastroenterology. 2013; 145(6):1392-403.e1-8.
16. González M A, Gonzalez-Rey E, Rico L, Büscher D, Delgado M. Adipose-derived mesenchymal stem cells alleviate experimental colitis by inhibiting inflammatory and autoimmune responses. Gastroenterology. 2009; 136(3):978-89.
17. Liang L, Dong C, Chen X, Fang Z, Xu J, Liu M, Zhang X, Gu DS, Wang D, Du W, Zhu D, Han Z C. Human umbilical cord mesenchymal stem cells ameliorate mice trinitrobenzene sulfonic acid (TNBS)-induced colitis. Cell Transplant. 2011; 20(9):1395-408.
18. Zhang Q, Shi S, Liu Y, Uyanne J, Shi Y, Le A D. Mesenchymal stem cells derived from human gingiva are capable of immunomodulatory functions and ameliorate inflammation-related tissue destruction in experimental colitis. J Immunol. 2009; 183(12):7787-98.
19. Chen Y, Yu Q, Hu Y, Shi Y. Current research and use of mesenchymal stem cells in the therapy of autoimmune diseases. Curr Stem Cell Res Ther. 2019; 14(7):579-82.
20. Zaher W, Harkness L, Jafari A, Kassem M. An update of human mesenchymal stem cell biology and their clinical uses. Arch Toxicol. 2014; 88(5):1069-82.
21. Duijvestein M, Vos A C, Roelofs H, Wildenberg M E, Wendrich B B, Verspaget H W, Kooy-Winkelaar E M, Koning F, Zwaginga J J, Fidder H H, Verhaar A P, Fibbe W E, van den Brink G R, Hommes D W. Autologous bone marrow-derived mesenchymal stromal cell treatment for refractory luminal Crohn's disease: results of a phase I study. Gut. 2010; 59(12):1662-9.
22. Nikolic A, Simovic Markovic B, Gazdic M, Randall Harrell C, Fellabaum C, Jovicic N, Djonov V, Arsenijevic N, L Lukic M, Stojkovic M, Volarevic V. Intraperitoneal administration of mesenchymal stem cells ameliorates acute dextran sulfate sodium-induced colitis by suppressing dendritic cells. Biomed Pharmacother. 2018; 100:426-32.
23. Fawzy S A, El-Din Abo-Elnou R K, Abd-El-Maksoud El-Deeb D F, Yousry Abd-Elkader M M. The possible role of mesenchymal stem cells therapy in the repair of experimentally induced colitis in male albino rats. Int J Stem Cells. 2013; 6(2): 92-103.
24. Hayashi Y, Tsuji S, Tsujii M, Nishida T, Ishii S, Nakamura T, Eguchi H, Kawano S. The transdifferentiation of bone-marrow-derived cells in colonic mucosal regeneration after dextran-sulfate-sodium-induced colitis in mice. Pharmacology. 2007; 80(4):193-9.
25 Anderson P, Souza-Moreira L, Morell M, Caro M, O'Valle F, Gonzalez-Rey E, Delgado M. Adipose-derived mesenchymal stromal cells induce immunomodulatory macrophages which protect from experimental colitis and sepsis. Gut. 2013; 62(8):1131-41.
26. Wu L W, Wang Y L, Christensen J M, Khalifian S, Schneeberger S, Raimondi G, Cooney D S, Lee W P, Brandacher G. Donor age negatively affects the immunoregulatory properties of both adipose and bone marrow derived mesenchymal stem cells. Transpl Immunol. 2014; 30(4): 122-7.
27 Kizilay Mancini Ö, Lora M, Shum-Tim D, Nadeau S, Rodier F, Colmegna I. A proinflammatory secretome mediates the impaired immunopotency of human mesenchymal stromal cells in elderly patients with atherosclerosis. Stem cells translational medicine. 2017; 6(4):1132-40.
28. Stolzing A, Jones E, McGonagle D, Scutt A. Age-related changes in human bone marrow-derived mesenchymal stem cells: consequences for cell therapies. Mech Ageing Dev. 2008; 129(3):163-73.
29. Sharma S, Mishra R, Bigham G E, Wehman B, Khan M M, Xu H, Saha P, Goo Y A, Datla S R, Chen L, Tulapurkar M E, Taylor B S, Yang P, Karathanasis S, Goodlett DR, Kaushal S. A deep proteome analysis identifies the complete secretome as the functional unit of human cardiac progenitor cells. Circ Res. 2017; 120(5):816-34.
30. Bury M, Fuller N, Clemons T, Sturm R, Morrison C, Lisy-Snow D, Nolan B, Tarcynzski C, Ayello E, Boyce A, Muckian B, Ahmad N, Hunter C, Karver M, Stupp SI, Sharma A K. Self-assembling nanofibers inhibit inflammation in a murine model of Crohn's-disease-like ileitis. Adv Therap February 2021
31. Bouma G, Strober W. The immunological and genetic basis of inflammatory bowel disease. Nat Rev Immunol. 2003; 3(7):521-33.
32. Behm B W, Bickston S J. Tumor necrosis factor-alpha antibody for maintenance of remission in Crohn's disease. Cochrane Database Syst Rev. 2008(1): CD 006893.
33. Zundler S, Becker E, Weidinger C, Siegmund B. Anti-adhesion therapies in inflammatory bowel disease-molecular and clinical aspects. Front Immunol. 2017; 8:891.
34. Reinisch W, Hommes D W, Van Assche G, Colombel J F, Gendre J P, Oldenburg B, Teml A, Geboes K, Ding H, Zhang L, Tang M, Cheng M, van Deventer S J, Rutgeerts P, Pearce T. A dose escalating, placebo controlled, double blind, single dose and multidose, safety and tolerability study of fontolizumab, a humanised anti-interferon gamma antibody, in patients with moderate to severe Crohn's disease. Gut. 2006; 55(8): 1138-44.
35. Miehsler W, Novacek G, Wenzl H, Vogelsang H, Knoflach P, Kaser A, Dejaco C, Petritsch W, Kapitan M, Maier H, Graninger W, Tilg H, Reinisch W, Hepatology ASoGa. A decade of infliximab: The Austrian evidence based consensus on the safe use of infliximab in inflammatory bowel disease. J Crohns Colitis. 2010; 4(3):221-56.
36. Vermeire S, Van Assche G, Rutgeerts P. Serum sickness, encephalitis and other complications of anti-cytokine therapy. Best Pract Res Clin Gastroenterol. 2009; 23(1):101-12.
37. Le Blanc K, Frassoni F, Ball L, Locatelli F, Roelofs H, Lewis I, Lanino E, Sundberg B, Bernardo ME, Remberger M, Dini G, Egeler RM, Bacigalupo A, Fibbe W, Ringdén O, Transplantation DCotEGfBaM. Mesenchymal stem cells for treatment of steroid-resistant, severe, acute graft-versus-host disease: a phase II study. Lancet. 2008; 371(9624):1579-86.
38. Sun L, Wang D, Liang J, Zhang H, Feng X, Wang H, Hua B, Liu B, Ye S, Hu X, Xu W, Zeng X, Hou Y, Gilkeson G S, Silver R M, Lu L, Shi S. Umbilical cord mesenchymal stem cell transplantation in severe and refractory systemic lupus erythematosus. Arthritis Rheum. 2010; 62(8):2467-75.
39. Yamout B, Hourani R, Salti H, Barada W, El-Hajj T, Al-Kutoubi A, Herlopian A, Baz E K, Mahfouz R, Khalil-Hamdan R, Kreidieh N M, El-Sabban M, Bazarbachi A. Bone marrow mesenchymal stem cell transplantation in patients with multiple sclerosis: a pilot study. J Neuroimmunol. 2010; 227(1-2):185-9.
40. García-Olmo D, García-Arranz M, Herreros D, Pascual I, Peiro C, Rodríguez-Montes J A. A phase I clinical trial of the treatment of Crohn's fistula by adipose mesenchymal stem cell transplantation. Dis Colon Rectum. 2005; 48(7):1416-23.
41. Watanabe S, Arimura Y, Nagaishi K, Isshiki H, Onodera K, Nasuno M, Yamashita K, Idogawa M, Naishiro Y, Murata M, Adachi Y, Fujimiya M, Imai K, Shinomura Y. Conditioned mesenchymal stem cells produce pleiotropic gut trophic factors. J Gastroenterol. 2014; 49(2):270-82.
42. Xie M, Qin H, Luo Q, He X, Lan P, Lian L. Comparison of adipose-derived and bone marrow mesenchymal stromal cells in a murine model of Crohn's disease. Dig Dis Sci. 2017; 62(1):115-23.
43. Zibandeh N, Genc D, Duran Y, Banzragch M, Sokwala S, Goker K, Atug O, AkkoçT. Human dental follicle mesenchymal stem cells alleviate T cell response in inflamed tissue of Crohn's patients. Turk J Gastroenterol. 2020; 31(5):400-9.
44. Lopez-Santalla M, Hervas-Salcedo R, Fernandez-Garcia M, Bueren J A, Garin M I. Cell therapy with mesenchymal stem cells induces an innate immune memory response that attenuates experimental colitis in the long term. J Crohns Colitis. 2020; 14(10):1424-35.
45. Baraniak P R, McDevitt T C. Stem cell paracrine actions and tissue regeneration. Regen Med. 2010; 5(1):121-43.
46. Saparov A, Ogay V, Nurgozhin T, Jumabay M, Chen WC. Preconditioning of human mesenchymal stem cells to enhance their regulation of the immune response. Stem Cells Int. 2016; 2016:3924858.
47. Badillo A T, Beggs K J, Javazon E H, Tebbets J C, Flake A W. Murine bone marrow stromal progenitor cells elicit an in vivo cellular and humoral alloimmune response. Biol Blood Marrow Transplant. 2007; 13(4):412-22.
48. Camp D M, Loeffler D A, Farrah D M, Borneman J N, LeWitt P A. Cellular immune response to intrastriatally implanted allogeneic bone marrow stromal cells in a rat model of Parkinson's disease. J Neuroinflammation. 2009; 6:17.
49. Gunasekaran M, Mishra R, Saha P, Morales D, Cheng W C, Jayaraman A R, Hoffman J R, Davidson L, Chen L, Shah A M, Bittle G, Fu X, Tulshyan A, Abdullah M, Kingsbury T, Civin C, Yang P, Davis M E, Bolli R, Hare J M, Sharma S, Kaushal S. Comparative efficacy and mechanism of action of cardiac progenitor cells after cardiac injury. iScience. 2022; 25(8):104656.
50. Ghia J E, Galeazzi F, Ford D C, Hogaboam CM , Vallance B A, Collins S. Role of M-CSF-dependent macrophages in colitis is driven by the nature of the inflammatory stimulus. Am J Physiol Gastrointest Liver Physiol. 2008; 294(3): G770-7.
51. Watanabe N, Ikuta K, Okazaki K, Nakase H, Tabata Y, Matsuura M, Tamaki H, Kawanami C, Honjo T, Chiba T. Elimination of local macrophages in intestine prevents chronic colitis in interleukin-10-deficient mice. Dig Dis Sci. 2003; 48(2):408-14.
52. Maggini J, Mirkin G, Bognanni I, Holmberg J, Piazzón I M, Nepomnaschy I, Costa H, Cañones C, Raiden S, Vermeulen M, Geffner J R. Mouse bone marrow-derived mesenchymal stromal cells turn activated macrophages into a regulatory-like profile. PLOS One. 2010; 5(2): e 9252.
53. Asami T, Ishii M, Fujii H, Namkoong H, Tasaka S, Matsushita K, Ishii K, Yagi K, Fujiwara H, Funatsu Y, Hasegawa N, Betsuyaku T. Modulation of murine macrophage TLR 7/8-mediated cytokine expression by mesenchymal stem cell-conditioned medium. Mediators Inflamm. 2013; 2013:264260.
54. Németh K, Leelahavanichkul A, Yuen P S, Mayer B, Parmelee A, Doi K, Robey P G, Leelahavanichkul K, Koller B H, Brown J M, Hu X, Jelinek I, Star R A, Mezey E. Bone marrow stromal cells attenuate sepsis via prostaglandin E(2)-dependent reprogramming of host macrophages to increase their interleukin-10 production. Nat Med. 2009; 15(1):42-9.
55. Wise A F, Williams T M, Kiewiet M B, Payne N L, Siatskas C, Samuel C S, Ricardo S D. Human mesenchymal stem cells alter macrophage phenotype and promote regeneration via homing to the kidney following ischemia-reperfusion injury. Am J Physiol Renal Physiol. 2014; 306(10):F1222-35.
56. Souza H S, Elia C C, Spencer J, MacDonald TT. Expression of lymphocyte-endothelial receptor ligand pairs, alpha4beta 7/MAdCAM-1 and OX40/OX40 ligand in the colon and jejunum of patients with inflammatory bowel disease. Gut. 1999 Dec; 45(6):856-63.
57. Rivera-Nieves J, Olson T, Bamias G, Bruce A, Solga M, Knight R F, Hoang S, Cominelli F, Ley K. L-selectin, alpha 4 beta 1, and alpha 4 beta 7 integrins participate in CD4+ T cell recruitment to chronically inflamed small intestine. J Immunol. 2005 February 15; 174(4):2343-52.
58. Rivera-Nieves J. Targeting beta-7 integrins for the treatment of inflammatory bowel disease. Hepatol (N Y). 2020 Jul; 16(7):367-369.
59. Simpson D L, Mishra R, Sharma S, Goh S K, Deshmukh S, Kaushal S. A strong regenerative ability of cardiac stem cells derived from neonatal hearts. Circulation. 2012;126(11 Suppl 1):S46-53.
60. Wehman B, Pietris N, Bigham G, Siddiqui O, Mishra R, Li T, Aiello E, Jack G, Wang W, Murthi S, Sharma S, Kaushal S. Cardiac progenitor cells enhance neonatal right ventricular function after pulmonary artery banding. Ann Thorac Surg. 2017; 104(6):2045-53.

1. A hierarchical metallic supra-nanostructure (HMSN) comprising branches with alternating nanocrystals and nano-linkers, wherein the nanocrystals comprise a first metal and the nano-linkers comprise a second metal having a lower reduction potential than the nanocrystals.

2. The hierarchical metallic supra-nanostructure of claim 1, wherein the nano-linkers are selectively dissolved in the presence of a reactive oxygen species (ROS).

3. The hierarchical metallic supra-nanostructure of claim 1, wherein the first metal is gold and/or the second metal is silver.

4. The hierarchical metallic supra-nanostructure of claim 3, wherein the first metal is gold and the second metal is silver.

5. The hierarchical metallic supra-nanostructure of claim 1, wherein the hierarchical metallic supra-nanostructure comprises primary branches and secondary branches extending from the primary branches.

6. The hierarchical metallic supra-nanostructure of claim 5, wherein the primary branches have a greater thickness than the secondary branches.

7. The hierarchical metallic supra-nanostructure of claim 1, wherein the hierarchical metallic supra-nanostructure has a particle size from 20 nm to 1000 nm.

8. The hierarchical metallic supra-nanostructure of claim 1, wherein the hierarchical metallic supra-nanostructure has an average particle size from about 20 nm to 800 nm.

9. The hierarchical metallic supra-nanostructure of claim 1, wherein the nanocrystals have a particle size from 1 nm to 10 nm.

10. The hierarchical metallic supra-nanostructure of claim 1, wherein the hierarchical metallic supra-nanostructure is two-dimensional.

11. The hierarchical metallic supra-nanostructure of claim 1 further comprising an agent conjugated with the hierarchical metallic supra-nanostructure.

12. The hierarchical metallic supra-nanostructure of claim 11, wherein the agent is a photosensitizer.

13. A method for preparing the hierarchical metallic supra-nanostructure of claim 1 comprising preparing a reaction mixture by adding a nanocrystal precursor and nano-linker precursor to a metal cholate solution; adding a reducing agent to the reaction mixture; and
preparing a nanoparticle suspension from the reduced reaction mixture.

14. The method of claim 13, wherein the nanocrystal precursor is AuCl, the nano-linker precursor is AgNO₃, the metal cholate is sodium cholate, the reducing agent is ascorbic acid, or any combination thereof.

15. The method of claim 14, wherein the nanoparticle suspension is prepared by adding a thiol-PEG-carboxylate to the reduced reaction mixture.

16. A method for treating a subject in need of a reactive oxygen species-mediated therapy, the method comprising administering the hierarchical metallic supra-nanostructure according to claim 1 and the reactive oxygen species-mediated therapy to the subject.

17. The method of claim 16, wherein administering the reactive oxygen species-mediated therapy comprises irradiating the subject.

18. The method of claim 16, wherein the subject is in need of a treatment for cancer.

19. The method of claim 18, wherein the method induces apoptosis, senescence, or cell cycle arrest in cancer cells.

20. (canceled)

21. A method for degrading the hierarchical metallic supra-nanostructure according to claim 1, the method comprising contacting the hierarchical metallic supra-nanostructure with a reactive oxygen species.

22-24. (canceled)
